(19) United States
(12) Patent Application Publication
(10) Pub. No.: US 2026/0159866 A1
(43) Pub. Date: Jun. 11, 2026

(54) METHODS FOR THE CHEMOENZYMATIC SYNTHESIS OF LOW MOLECULAR WEIGHT HEPARIN FROM LOW MOLECULAR WEIGHT HEPAROSAN

(71) Applicants: Yanlei YU, Hangzhou City, Zhejiang Province (CN); Li FU, Mason, OH (US); Hong WANG, Hangzhou Shi, Zhejiang Sheng (CN); RENSSELAER POLYTECHNIC INSTITUTE, Troy, NY (US); OTSUKA PHARMACEUTICAL FACTORY, INC., Naruto, Tokushima (JP)

(72) Inventors: Robert John LINHARDT, Albany, NY (US); Jonathan Seth DORDICK, Schenectady, NY (US); Yanlei YU, Hangzhou City (CN); Li FU, Mason, OH (US); Peng HE, Troy, NY (US); Ke XIA, Latham, NY (US); Sony VARGHESE, Latham, NY (US); Fuming ZHANG, Watervliet, NY (US)

(73) Assignees: RENSSELAER POLYTECHNIC INSTITUTE, Troy, NY (US); OTSUKA PHARMACEUTICAL FACTORY, INC., Naruto, Tokushima (JP)

(21) Appl. No.: 18/707,500

(22) PCT Filed: Nov. 4, 2022
(86) PCT No.: PCT/US2022/048928

§ 371 Date: May 3, 2024

Related U.S. Application Data
(60) Provisional application No. 63/276,212, filed on Nov. 5, 2021.
(60) Provisional application No. 63/310,410, filed on Feb. 15, 2022.

(51) Int. Cl. C12P 19/04; C12N 9/10; C12N 9/90; C12P 19/24

(52) U.S. Cl. CPC C12P 19/04; C12N 9/13; C12N 9/90; C12P 19/24; C12Y 208/02; C12Y 501/03

(57) ABSTRACT

Low molecular weight heparin (LMWH) suitable for equivalent use to USP enoxaparin sodium is prepared from a starting material isolated from engineered E. coli K5 capsular polysaccharide, e.g., E. coli K5 heparosan. The E. Coli CPS is treated with acids to remove 3-deoxy-D-manno oct-2-ulosonic acid (Kdo) residues, and further hydrolyzed via alkali treatment to form low molecular weight N-sulfo, N-acetyl heparosan (LMW-NSNAH) having molecular weight and N-acetylation comparable to enoxaparin. The LMW-NSNAH is converted to LMWH via a series of enzymatic modifications by C5-epimerase, 2-O-, 6-O-, and 3-O-sulfotransferases. Compositions including the LMWH are prepared without the use of porcine-derived heparin, and thus benefit from better source material availability, better control of manufacturing processes, reduced concerns about contamination, adulteration or animal virus, or impurities. Further, the LMWH product is demonstrated to be structurally and functionally comparable traditional pharmaceutical LMWHs.

### CROSS REFERENCE TO RELATED APPLICATION(S)

This application claims the benefit of U.S. Provisional Application Nos. 63/276,212, filed Nov. 5, 2021, and 63/310,410, filed Feb. 15, 2022, which are incorporated by reference as if disclosed herein in their entireties.

### STATEMENT REGARDING FEDERALLY SPONSORED RESEARCH AND DEVELOPMENT

This invention was made with U.S. Government support under Grant Number DMR-1933525 awarded by the National Science Foundation. The United States Government has certain rights in the invention.

### BACKGROUND

Heparin products are widely used clinical anticoagulants used in the practice of modern medicine. Low molecular weight heparins (LMWHs) are currently prepared by the controlled chemical or enzymatic depolymerization of unfractionated heparins (UFHs) that are extracted from animal tissues. In many clinical applications, LMWHs have displaced UFHs and currently comprise over 60% of the heparin market. In the past, extensive efforts have been made to prepare bioengineered UFHs relying on a chemoenzymatic process to address concerns about animal sourced UFHs.

Heparin is typically prepared from animal tissues rich in heparin proteoglycan, primarily from porcine intestines. Heparin is a linear highly sulfated polysaccharide found covalently attached to the core protein serglycin as a proteoglycan and stored in intracellular granules of mast cells. It is composed of a repeating disaccharide unit comprised of β-D-glucuronic acid (GlcA) or α-L-iduronic acid (IdoA) 1, 4-glycosidically linked to D-glucosamine (GlcN). Unlike the synthesis of DNA and proteins, the biosynthesis of heparin is not template driven, and thus the resulting polysaccharides are heterogeneous in length and substitution pattern. Heparin biosynthesis in certain animal cells begins in the endoplasmic reticulum involving formation of a tetrasaccharide linker (D-xylose (Xyl)-D-galactose (Gal)-Gal-GlcA) that tethers to a serine residue of its core protein. Chain polymerization next takes place through formation of a repeating disaccharide building block of N-acetyl-α-D-glucosamine (GlcNAc) 1,4-linked GlcA driven by two polymerases known as exostosin glycosyltransferase (EXT) 1 and EXT 2, forming heparosan, the backbone of heparin.

Referring now to FIG. 1, heparosan is a linear chain of repeating disaccharide units of [→4) GlcA (1→4) GlcNAc (1→]ₙ. Subsequent modification of this backbone takes place through de-N-acetylation and N-sulfation, C5-epimerization, and a series 3′-phosphoadenosine 5′-phosphosulfate (PAPS)-dependent O-sulfation reactions all occurring in the Golgi compartment. These reactions are catalyzed by N-deacetylase/N-sulfotransferase (NDST) to form N-sulfo-α-D-glucosamine (GlcNS) residues, C5-epimerase (Epi), converting GlcA residues to L-iduronic acid (IdoA), and 2-O, 6-O, 3-O-sulfotransferases (STs) that transfer sulfo groups to the polysaccharide chain. Pharmaceutical heparin is polydisperse and heterogeneous, having an average molecular weight of 18-20 kDa.

As mentioned above, LMWHs are currently produced by either controlled chemical or enzymatic depolymerization of UFH. LMWHs have several advantages over UFH for therapeutic anticoagulation including high subcutaneous bioavailability and a more predictable pharmacokinetic profile, a longer plasma half-life, and lower incidences of heparin-induced thrombocytopenia (HIT). Commercially available LMWHs are polydisperse, fractionated heparins with average molecular weights ranging from 3-8 kDa. For example, enoxaparin (˜4,500 Da) is produced using benzylation followed by alkaline hydrolysis, dalteparin (˜6,000 Da) is derived from controlled nitrous acid depolymerization, and tinzaparin (˜6,500 Da) is prepared by controlled heparinase digestion. Among the three, enoxaparin (Lovenox®) produced by Sanofi has the major share of the worldwide LMWH market and the most extensive clinical evidence of efficacy and safety in various applications, and hence, has the broadest range of therapeutic indications. Recently, patent rights and supplementary protection certificates of originator enoxaparin have expired. The approval of generic forms of enoxaparin by the U. S. Food and Drug Administration (FDA) in 2010 has reduced the drug price, making LMWHs available to broader patient populations. However, the quality and supply of LMWHs rely on the quality of animal-derived heparin. There is growing concern about a shortage of porcine heparin, and the supply chain of heparin, and thus of LMWH, is under threat of impurities, contamination, and adulteration. To this end, effort has been directed towards developing and improving techniques and approaches towards the synthesis of UFH and LMWH.

The chemoenzymatic synthesis of UFH has become possible with successful expression of recombinant heparin biosynthetic enzymes including glycosyltransferases, C5-Epi, and 2-, 6-, 3-OSTs. A chemoenzymatic approach closely mimics the heparin biosynthetic pathway. Bioengineered UFH preparation starts with the Escherichia coli K5 capsular polysaccharide (CPS), heparosan, as the starting material. Chemical de-N-acetylation and N-sulfonation of heparosan affords N-sulfoheparosan that is subsequently modified using C5-Epi and 2-, 6-, 3-OSTs. This bioengineered UFH has shown chemical and biological equivalence to pharmaceutical porcine heparin.

A homogeneous, monodisperse, fondaparinux-like, ultra-LMWH has been chemoenzymatically synthesized from uridine-5′-diphosphate (UDP)-sugar donors and a heparosan-derived disaccharide acceptor using N-acetyl glucosaminyltransferase (KfiA) and heparosan synthase (pmHS2). Furthermore, a single targeted structure of a homogeneous dodecasaccharide LMWH has also been synthesized and demonstrated to be a viable candidate to replace LMWHs in thromboprophylaxis. These chemoenzymatic processes rely on the use of expensive UDP-sugar donors to iteratively synthesize homogeneous molecular species.

### SUMMARY

Aspects of the present disclosure are directed to a method of making low molecular weight heparin (LMWH). In some embodiments, the method includes providing an amount of heparosan; contacting the heparosan with one or more acids to form acid-treated heparosan; converting the acid-treated heparosan by depolymerization and de-N-acetylation thereof to form low molecular weight N-sulfo, N-acetyl heparosan (LMW-NSNAH); and enzymatically converting the LMW-NSNAH to LMWH. In some embodiments, the heparosan is an E. coli capsular polysaccharide. In some embodiments, the heparosan is synthesized via an engineered strain of E. coli K5.

In some embodiments, contacting the heparosan with one or more acids to form acid-treated heparosan includes removing 3-deoxy-D-manno oct-2-ulosonic acid (Kdo) residues from the heparosan via acid hydrolysis. In some embodiments, converting the acid-treated heparosan to LMW-NSNAH includes hydrolysis of the acid-treated heparosan via treatment of the acid-treated heparosan with one or more bases; treatment of the acid-treated heparosan with one or more additional acids; contacting the acid-treated heparosan with one or more enzymes; or combinations thereof. In some embodiments, converting the acid-treated heparosan by depolymerization and de-N-acetylation thereof to LMW-NSNAH further includes re-acetylating the acid-treated heparosan after de-N-acetylation thereof and N-sulfating the acid-treated heparosan to obtain LMW-NSNAH.

In some embodiments, re-acetylating the acid-treated heparosan after de-N-acetylation thereof includes contacting the acid-treated heparosan with acetic anhydride. In some embodiments, N-sulfating the acid-treated heparosan to obtain LMW-NSNAH includes contacting the acid-treated heparosan with trimethylamine sulfur trioxide, pyridine sulfur trioxide, or combination thereof. In some embodiments, re-acetylating the acid-treated heparosan after de-N-acetylation thereof includes contacting the acid-treated heparosan with about 53 μM/L acetic anhydride. In some embodiments, N-sulfating the acid-treated heparosan to obtain LMW-NSNAH includes contacting the acid-treated heparosan with about 76 mM/L trimethylamine sulfur trioxide.

In some embodiments, enzymatically converting the LMW-NSNAH to LMWH includes contacting the LMW-NSNAH with C5-Epi and 2-OST to form N-sulfo, N-acetyl, 2-O-sulfo IdoA-including heparosan (NSNA2SH). In some embodiments, enzymatically converting the LMW-NSNAH to LMWH includes contacting the NSNA2SH with 6-O-sulfotransferase-3, 6-O-sulfotransferase-1, or combinations thereof to form N-sulfo, N-acetyl, 2-O-sulfo, 6-O-sulfo IdoA-including heparosan (NSNA2S6SH). In some embodiments, enzymatically converting the LMW-NSNAH to LMWH includes contacting the NSNA2S6SH with 3-O-sulfotransferase-1 to form LMWH.

In some embodiments, the LMW-NSNAH has a molecular weight between about 3,800 and about 4,500 daltons. In some embodiments, the LMW-NSNAH includes between about 10% to about 15% N-acetyl groups. In some embodiments, the LMWH is a heterogeneous, polydisperse form of enoxaparin with an anti-factor Xa between about 90 and about 125 IU/mg.

Aspects of the present disclosure are directed to an intermediate LMW-NSNAH, the LMW-NSNAH being produced by a method including providing an amount of heparosan, wherein the heparosan is an E. coli capsular polysaccharide; contacting the heparosan with one or more acids to remove Kdo residues from the heparosan via hydrolysis to form acid-treated heparosan; and converting the acid-treated heparosan by depolymerization and de-N-acetylation thereof to form LMW-NSNAH. In some embodiments, the LMW-NSNAH has a molecular weight and a ratio of N-sulfo groups and N-acetyl groups such that enzymatic treatment thereof with a C5-epimerase and at least one sulfotransferase yields a final product with molecular weight and chemical properties consistent with animal-derived enoxaparin. In some embodiments, the LMW-NSNAH has a molecular weight between about 3,800 and about 4,500 daltons. In some embodiments, the LMW-NSNAH includes between about 10% to about 15% N-acetyl groups.

In some embodiments, converting the acid-treated heparosan to LMW-NSNAH includes hydrolysis via treatment of the acid-treated heparosan with one or more bases; treatment of the acid-treated heparosan with one or more additional acids; contacting the acid-treated heparosan with one or more enzymes; or combinations thereof. In some embodiments, converting the acid-treated heparosan by depolymerization and de-N-acetylation thereof to LMW-NSNAH further includes adding the acid-treated heparosan to a reaction medium including methanol, anhydrous sodium carbonate, and about 53 μM/L acetic anhydride to form re-acetylated heparosan and adding the re-acetylated heparosan to a reaction medium including anhydrous sodium carbonate and about 76 mM/L trimethylamine sulfur trioxide to obtain LMW-NSNAH.

Aspects of the present disclosure are directed to a composition including LMWH, wherein the LMWH is prepared via enzymatic conversion of LMW-NSNAH prepared from E. coli capsular polysaccharide.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chemical structure for heparosan;

FIG. 2 is a chart of a method of making low molecular weight heparin (LMWH) according to some embodiments of the present disclosure;

FIG. 3 is a graph showing ¹H NMR analysis of 3-deoxy-D-manno oct-2-ulosonic acid (Kdo) removal from low molecular weight N-sulfo, N-acetyl heparosan (LMW-NSNAH);

FIG. 4A is a graph showing molecular weight analysis of chemobiosynthetic LMW-NSNAH by gel permeation chromatography (GPC);

FIG. 4B is a graph showing molecular weight analysis of chemobiocatalytic LMWH by GPC;

FIG. 5A is a graph showing NS2S conversion by 2-O-sulfotransferase and C5-epimerase reaction during enzymatic synthesis of chemobiosynthetic LMWH according to some embodiments of the present disclosure;

FIG. 5B is a graph showing Tris conversion by 6-O-sulfotransferase reaction during enzymatic synthesis of chemobiosynthetic LMWH according to some embodiments of the present disclosure;

FIG. 5C is a graph 3S conversion by 3-O-sulfotransferase reaction during enzymatic synthesis of chemobiosynthetic LMWH according to some embodiments of the present disclosure as evidenced anti-Xa activity;

FIG. 6 is a table of disaccharide structures of chemobiosynthetic LMWH according to some embodiments of the present disclosure and its intermediates identified via treatment with heparin lyases I, II and III;

FIG. 7A is a graph showing disaccharide spectrum analysis of chemobiosynthetic LMWH according to some embodiments of the present disclosure by strong anion exchange high-performance liquid chromatography (SAX-HPLC);

FIG. 7B is a graph showing tetrasaccharide spectrum analysis of chemobiosynthetic LMWH according to some embodiments of the present disclosure by SAX-HPLC;

FIG. 8A is a graph showing disaccharide composition analysis of chemobiosynthetic LWMH according to some embodiments of the present disclosure;

FIG. 8B is a graph showing tetrasaccharide composition analysis of chemobiosynthetic LWMH according to some embodiments of the present disclosure;

FIG. 9 portrays chemical structures for 5 3-O-sulfated containing tetrasaccharide structures from chemobiosynthetic LWMH according to some embodiments of the present disclosure;

FIG. 10A is a graph showing ¹H NMR of enoxaparin and chemobiosynthetic LMWH according to some embodiments of the present disclosure;

FIG. 10B is a graph showing ¹³C NMR analysis of enoxaparin and chemobiocatalytic LMWH according to some embodiments of the present disclosure;

FIGS. 11A-11B are graphs showing surface plasmon resonance (SPR) sensorgrams of antithrombin III (AT) binding to heparin surface competing with enoxaparin and chemobiocatalytic LMWH according to some embodiments of the present disclosure respectively;

FIG. 11C is a graph showing IC₅₀ calculation of enoxaparin and chemobiocatalytic LMWH according to some embodiments of the present disclosure using AT inhibition data;

FIGS. 11D-11E are graphs showing SPR sensorgrams of platelet factor IV (PF4) binding to heparin surface competing with enoxaparin and chemobiocatalytic LMWH according to some embodiments of the present disclosure respectively; and

FIG. 11F is a graph showing IC₅₀ calculation of enoxaparin and chemobiocatalytic LMWH according to some embodiments of the present disclosure using PF4 inhibition data.

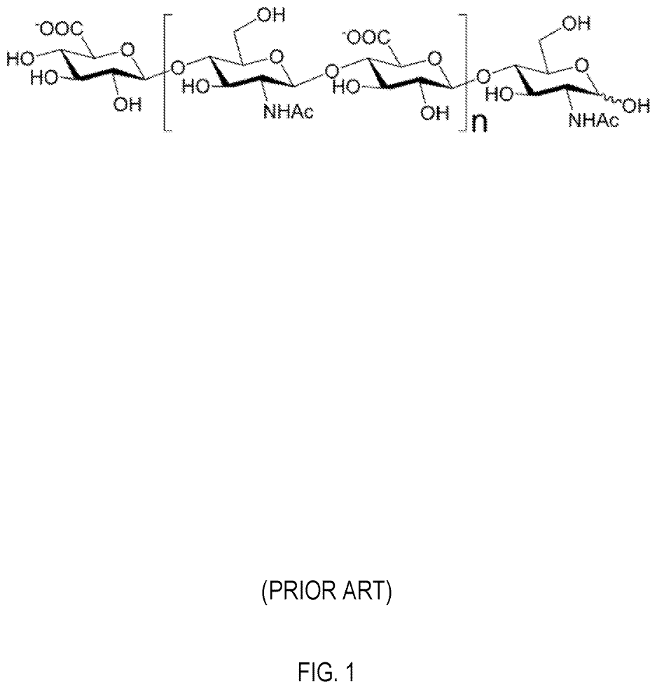

### DETAILED DESCRIPTION

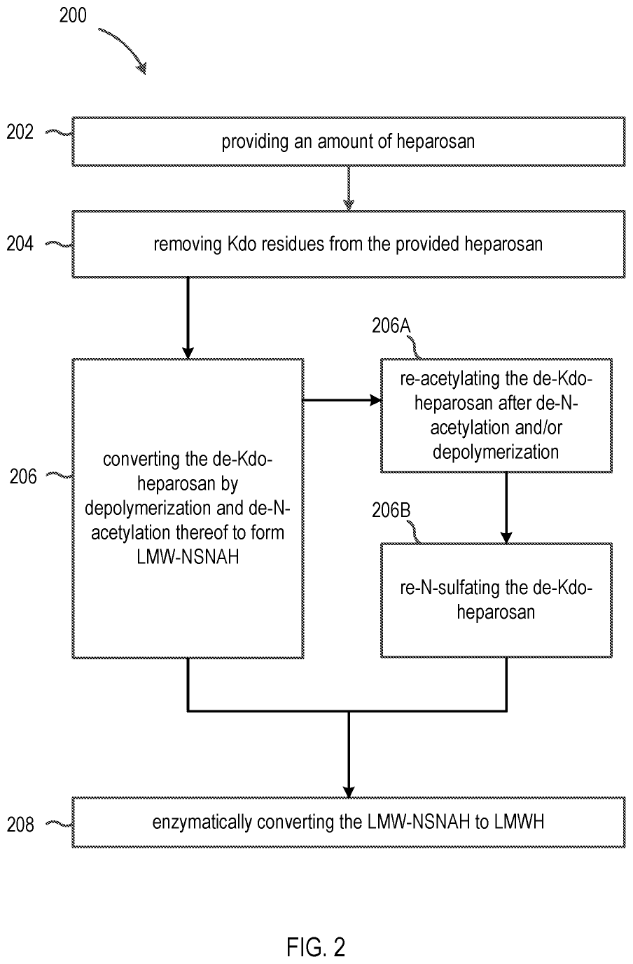

Referring now to FIG. 2, some embodiments of the present disclosure are directed to a method 200 of making low molecular weight heparin (LMWH), also referred to herein as “chemobiosynthetic” or “chemobiocatalytic” LMWH. In some embodiments, at 202, an amount of heparosan is provided. In some embodiments, the heparosan is synthesized by a bacterial source, i.e., one or more bacteria. In some embodiments, the heparosan is isolated from the one or more bacteria for use in the steps of method 200, i.e., method 200 occurs extracellularly. In some embodiments, the heparosan secreted by the bacterial source and subsequently isolated therefrom for use in method 200. In some embodiments, the heparosan is collected for use in method 200 via any suitable process after lysis of the bacterial source to release the heparosan. In some embodiments, at least some steps of method 200 occur intracellularly, i.e., within the bacterial source itself. In some embodiments, the heparosan is provided 202 to a reaction vessel, wherein at least one of the subsequent steps in method 200 is performed.

In some embodiments, the bacterial source is any suitable wild-type or engineered bacteria. In some embodiments, the bacterial source includes an Escherichia coli (E. coli) strain. In some embodiments, the bacterial source includes E. coli K5. In some embodiments, the bacterial source includes an engineered strain of E. coli K5. In some embodiments, the engineered strain of E. coli K5 has had fructosyl transferase removed.

In some embodiments, the heparosan is E. coli capsular polysaccharide (CPS). Without wishing to be bound by theory, the heparosan isolated from the bacterial source, e.g., engineered strains of E. coli K5 with fructosyl transferase removed, for use in method 200 is an acidic CPS. As discussed above, heparosan is a linear chain of repeating structure→4)-β-GlcA) (1→4)-α-GlcNAc (1→. In some embodiments, the heparosan for use in method 200 has an average molecular weight between about 35 kDa and about 65 kDa. In some embodiments, the heparosan has an average molecular weight between about 45 kDa and about 55 kDa. In some embodiments, the heparosan has an average molecular weight between about 48 kDa and about 52 kDa. In exemplary embodiments, the heparosan CPS provided at step 202 can have an average molecular weight of 49 kDa, much larger than the molecular weight of commercial UFH and LMWH.

In some embodiments, at 204, 3-deoxy-D-manno oct-2-ulosonic acid (Kdo) residues are removed from the provided heparosan. In some embodiments, the Kdo residues are removed from the heparosan via hydrolysis. In some embodiments, the Kdo residues are removed from the heparosan via acid hydrolysis. In some embodiments, the heparosan is contacted with one or more acids to remove the Kdo residues and form acid-treated heparosan via the acid hydrolysis. In some embodiments, the one or more acids include any acid or combination of acids suitable for removing the Kdo residues without degrading the heparosan to such an extent that it can no longer be enzymatically converted to heparin, as will be discussed in greater detail below. In some embodiments, the one or more acids includes hydrochloric acid (HCl).

In some embodiments, at 206, the heparosan with Kdo residues removed (also referred to herein as “de-Kdo-heparosan”) is converted by depolymerization and de-N-acetylation thereof to form low molecular weight N-sulfo, N-acetyl heparosan (LMW-NSNAH). In an exemplary embodiment, the one or more acids utilized at step 204 also act to reduce the heparosan molecular weight, de-N-acetylate the heparosan, or combinations thereof. In some embodiments of step 206, it is the acid-treated heparosan that is converted by depolymerization and de-N-acetylation thereof to form the LMW-NSNAH. In some embodiments, the de-Kdo-heparosan is de-N-acetylated and depolymerized to obtain a low molecular weight form of heparosan, e.g., LMW-NSNAH, having an average molecular weight between about 3,000 and about 10,000 daltons. In some embodiments, the de-Kdo-heparosan is de-N-acetylated and depolymerized to obtain LMW-NSNAH having an average molecular ranging from about 4,000 to about 7,000 daltons. In some embodiments, the de-Kdo-heparosan is de-N-acetylated and depolymerized to obtain LMW-NSNAH having an average molecular ranging from about 3,800 to about 4,500 daltons. In some embodiments, the de-Kdo-heparosan is de-N-acetylated and depolymerized to obtain between about 85% and about 90% de-N-acetylation.

In some embodiments, converting 206 the de-Kdo-heparosan, e.g., the acid-treated heparosan, by depolymerization and de-N-acetylation thereof occurs via hydrolysis. In some embodiments, the hydrolysis is the result of treatment of the de-Kdo-heparosan with one or more bases, one or more additional acids, one or more enzymes, or combinations thereof. In some embodiments, the one or more bases includes an alkali composition, e.g., includes one or more alkali metals. In some embodiments, the one or more bases includes a hydroxide. In some embodiments, the one or more bases includes sodium hydroxide (NaOH). In some embodiments, the one or more bases have a concentration between about IN and about 3N. In some embodiments, the one or more bases have a concentration of about 2N. In some embodiments, the one or more enzymes include endo-β-glucuronidase.

As discussed above, in some embodiments, the de-Kdo-heparosan is depolymerized to reach an average molecular weight between about 3,000 and about 10,000 daltons. In some embodiments, the de-Kdo-heparosan is depolymerized to reach an average molecular weight between about 4,000 and about 7,000 daltons. In some embodiments, the de-Kdo-heparosan is depolymerized to reach an average molecular weight between about 3,800 and about 4,500 daltons. In some embodiments, the de-Kdo-heparosan is de-N-acetylated to reach about 10% to about 15% of N-acetyl groups on the de-Kdo-heparosan. In some embodiments, reaction temperature (55, 60, 65, and 70° C.) and time (24, 48, 72 and 96 h) can be used for converting step 206. In an exemplary embodiment, after a 48 h reaction time at 65° C., the average molecular weight of the de-Kdo-heparosan decreased to 3.9 kDa as determined by GPC.

In some embodiments, converting 206 the de-Kdo-heparosan includes re-acetylating 206A the de-Kdo-heparosan after de-N-acetylation and/or depolymerization thereof. In some embodiments, the depolymerized heparosan is at least partially re-acetylated. In some embodiments, re-acetylating 206A the de-Kdo-heparosan includes contacting the de-Kdo-heparosan with acetic anhydride. In some embodiments, re-acetylating 206A includes adding methanol, anhydrous sodium carbonate, and acetic anhydride. In some embodiments, the amount of acetic anhydride added is sufficient to reach about 10% to about 15% of N-acetyl groups on the de-Kdo-heparosan. In some embodiments, the concentration of the acetic anhydride is about between about 40 μM/L and about 60 μM/L. In some embodiments, the concentration of the acetic anhydride is about between about 45 μM/L and about 55 μM/L. In some embodiments, the concentration of the acetic anhydride is about between about 50 μM/L and about 55 μM/L. In some embodiments, the concentration of the acetic anhydride is about 53 μM/L. In some embodiments, the de-Kdo-heparosan is contacted with acetic anhydride a plurality of times. In some embodiments, the de-Kdo-heparosan is contacted with acetic anhydride at least 4 times at predetermined intervals. In some embodiments, the intervals are regular. In some embodiments, the intervals are irregular. In some embodiments, the intervals are between about 10 minutes and about 30 minutes. In some embodiments, the intervals are about 20 minutes.

In some embodiments, converting 206 the de-Kdo-heparosan further includes N-sulfating 206B the de-Kdo-heparosan. In some embodiments, N-sulfating 206B the de-Kdo-heparosan obtains LMW-NSNAH. In some embodiments, N-sulfating 206B the de-Kdo-heparosan includes contacting the de-Kdo-heparosan, e.g., acid-treated heparosan, with trimethylamine sulfur trioxide, pyridine sulfur trioxide, or combinations thereof. In some embodiments, the heparosan is N-sulfated 206B by adding an equal portion of anhydrous sodium carbonate, and trimethylamine sulfur trioxide. In some embodiments, the concentration of the trioxide reactant, e.g., trimethylamine sulfur trioxide, pyridine sulfur trioxide, etc., is between about 60 mM/L and about 90 mM/L. In some embodiments, the concentration of the trioxide reactant is between about 70 mM/L and about 80 mM/L. In some embodiments, the concentration of the trioxide reactant is about 76 mM/L.

In some embodiments, the LMW-NSNAH has a molecular weight between about 3,000 and about 10,000 daltons. In some embodiments, the LMW-NSNAH has a molecular weight between about 4,000 and about 7,000 daltons. In some embodiments, the LMW-NSNAH has a molecular weight between about 3,800 and about 4,500 daltons. In some embodiments, the LMW-NSNAH includes between about 10% to about 15% N-acetyl groups.

At 208, the LMW-NSNAH is enzymatically converted to LMWH. In some embodiments, enzymatic conversion 208 occurs via one or more sequential enzymatic treatments, each treatment including one or more enzymes. In some embodiments, enzymatically converting 208 the LMW-NSNAH to LMWH includes contacting the LMW-NSNAH with C5-epimerase (C5-Epi) and 2-O-sulfotransferase (2-OST) to form N-sulfo, N-acetyl, 2-O-sulfo IdoA-including heparosan (NSNA2SH). In some embodiments, enzymatically converting 208 the LMW-NSNAH to LMWH includes contacting the NSNA2SH with one or more sulfotransferases. In some embodiments, enzymatically converting 208 the LMW-NSNAH to LMWH includes contacting the NSNA2SH with 6-O-sulfotransferase-3 (6-OST-3), 6-O-sulfotransferase-1 (6-OST-1), or combinations thereof, to form N-sulfo, N-acetyl, 2-O-sulfo, 6-O-sulfo IdoA-including heparosan (NSNA2S6SH). In some embodiments, enzymatically converting 208 the LMW-NSNAH to LMWH includes contacting the NSNA2S6SH with 3-O-sulfotransferase-1 (3-OST) to form LMWH. In some embodiments, the LMWH is a heterogeneous, polydisperse form of enoxaparin with an anti-factor Xa between about 90 and about 125 IU/mg. In some embodiments, an amount of 1,6-anhydromannose including chains are introduced to the LMWH.

Some embodiments of the present disclosure are directed to the intermediate LMW-NSNAH. As discussed above, the intermediate LMW-NSNAH is the result of one or more processing steps to a heparosan starting material synthesized by a bacterial source. Sourcing the heparosan starting material from such bacteria for subsequent conversion to a heparin product provides numerous advantages over animal-derived heparin products, e.g., material availability, purity, etc., and the methods of the present disclosure ensure that the bacteria-derived heparosan is converted to an intermediate LMW-NSNAH and subsequently a LMWH that is functionally equivalent of that animal-derived heparin.

In some embodiments of the methods of making LMW-NSNAH, an amount of heparosan is provided. As discussed above, in some embodiments, the heparosan is E. coli CPS. In some embodiments, the heparosan is contacted with one or more acids to remove Kdo residues from the heparosan via hydrolysis to form acid-treated heparosan. In some embodiments, the acid-treated heparosan, now free of Kdo upon acid hydrolysis, can then be isolated from the rest of the reaction medium using a suitably sized and configured separation membrane, e.g., with 1 kDa molecular weight cut-off.

A major difference between heparosan intermediate in animals and the heparosan in CPS for use in the methods of the present disclosure is the acceptors on which they are biosynthesized. In animals, the heparosan is assembled on an acceptor corresponding to the tetrasaccharide linkage region (Xyl-Gal-Gal-GlcA) attached to serine residue of the core protein serglycin. However, the biosynthesis of heparosan CPS initiates on a glycolipid acceptor, which is composed of multiple, linked Kdo residues.

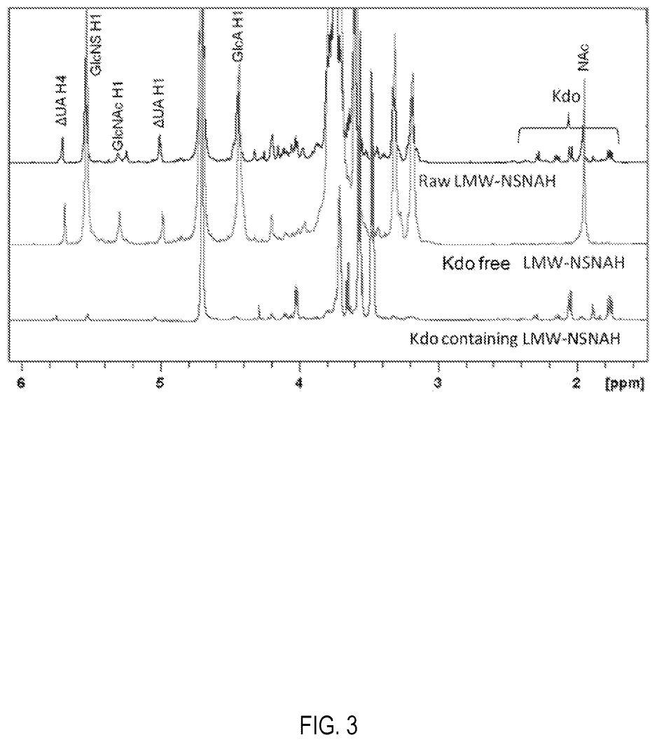

Referring now to FIG. 3, as discussed above in embodiments of the present disclosure at step 204, the glycolipid terminus (including Kdo residues) is removed before additional LMWH-synthesis steps, e.g., steps 206-208, since it is not found in porcine-derived LMWH products. Reaction conditions from step 204 work to remove the Kdo, but can also hydrolyze N-acetyl groups and reduce heparosan molecular weight. Using ¹H NMR and GPC analysis, it was determined that treating heparosan, in an exemplary embodiment from E. coli K5 (with fructosyl transferase removed) with HCl at pH=1 for 1 h at 90° C., releases Kdo without modifying heparosan chains. The Kdo signals observed at 1.5-2.5 ppm in 1H NMR were missing in the retentate indicating that the Kdo had been successfully removed.

In some embodiments, the acid-treated heparosan is then converted by depolymerization and de-N-acetylation thereof to form the LMW-NSNAH. In some embodiments, converting the acid-treated heparosan to LMW-NSNAH includes hydrolysis via treatment of the acid-treated heparosan with one or more bases, treatment of the acid-treated heparosan with one or more additional acids, contacting the acid-treated heparosan with one or more enzymes, or combinations thereof. In some embodiments, converting the acid-treated heparosan to LMW-NSNAH includes adding the acid-treated heparosan to a reaction medium including methanol, anhydrous sodium carbonate, and about 53 μM/L acetic anhydride to form re-acetylated heparosan.

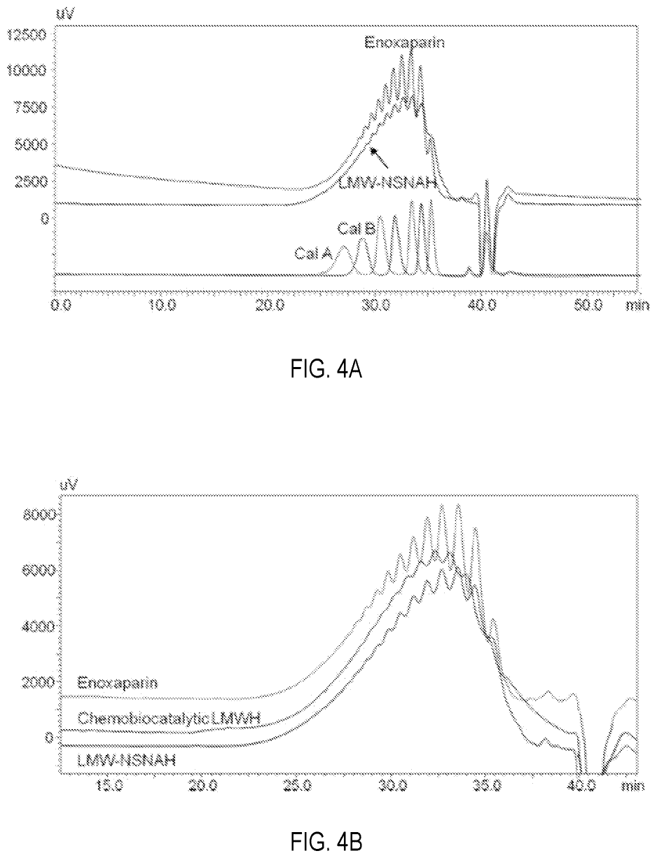

Referring now to FIG. 4A-4B, the chemical de-N-acetylation of heparosan consistent with embodiments of the present disclosure, e.g., by base hydrolysis, results in partial (or complete) removal of N-acetyl groups of the GlcNAc residues and polysaccharide chain depolymerization through β-elimination. In exemplary embodiments, due to the reaction conditions in the de-N-acetylation from step 206 above, no acetyl group (100% de-N-acetylation) was found based on NMR analysis. The heparosan was then re-acetylated, e.g., at step 206A, by adding an amount of acetic anhydride after base treatment and prior to N-sulfation, e.g., at step 206B. In some embodiments, converting the acid-treated heparosan to LMW-NSNAH includes adding the re-acetylated heparosan to a reaction medium including anhydrous sodium carbonate and about 76 mM/L trimethylamine sulfur trioxide to N-sulfate the re-acetylated heparosan and obtain LMW-NSNAH.

Referring specifically to FIG. 4A, in such an exemplary embodiment, 146 mg low molecular weight N-sulfo, N-acetyl heparosan was obtained from 1 g of acid-treated heparosan, with a molecular weight of 4,200 Da. ¹H NMR analysis of the 5.31 ppm peak, corresponding to the GlcNAc residue, and the 5.55 ppm peak, corresponding to the GlcNS residue, afforded an N-acetyl/N-sulfo ratio ranging from 10% to 15%, affording a product matching the United States Pharmacopeia (USP) criteria for enoxaparin. In some embodiments, the LMW-NSNAH has a molecular weight and a ratio of N-sulfo groups and N-acetyl groups such that enzymatic treatment thereof with a C5-Epi and sulfotransferase, e.g., 2-OST, 6-OST-1, 6-OST-3, 3-OST, etc., or combinations thereof, yields a final product with molecular weight and chemical properties consistent with animal-derived enoxaparin. As discussed above, in some embodiments, the LMW-NSNAH has a molecular weight between about 3,000 and about 10,000 daltons. In some embodiments, the LMW-NSNAH has a molecular weight between about 4,000 and about 7,000 daltons. In some embodiments, the LMW-NSNAH has a molecular weight between about 3,800 and about 4,500 daltons. In some embodiments, the LMW-NSNAH includes between about 10% to about 15% N-acetyl groups.

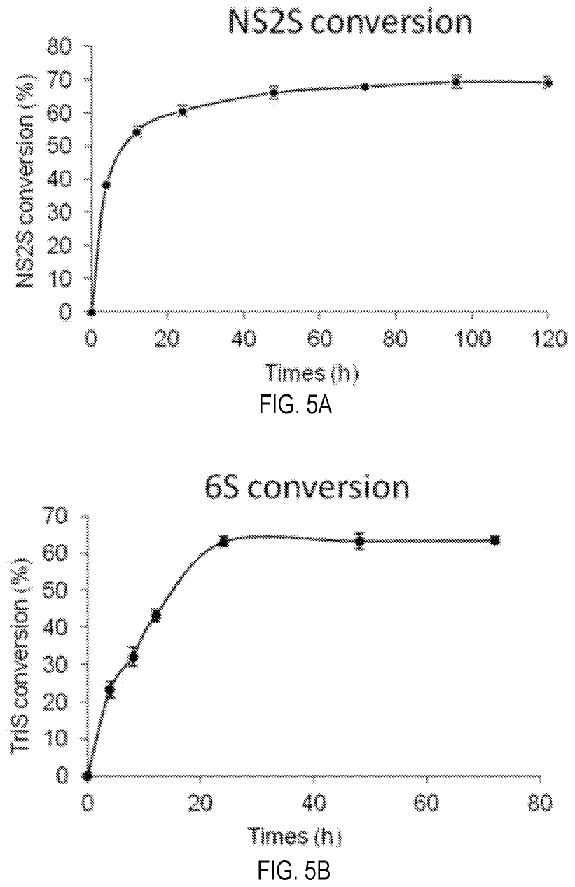

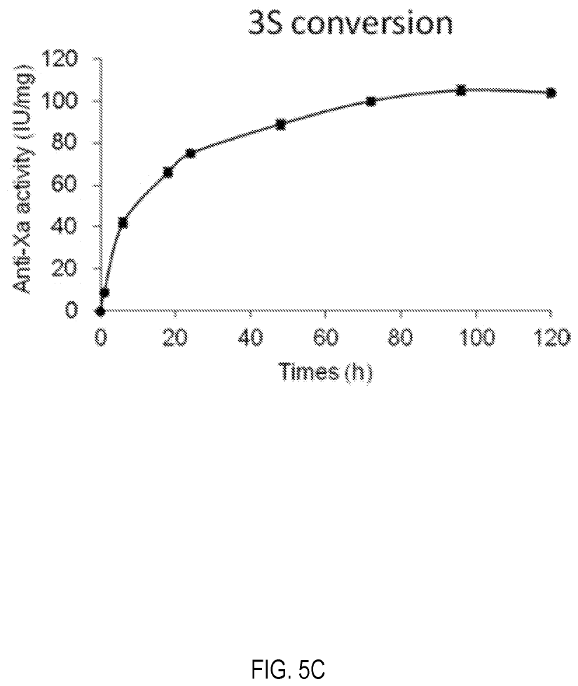

Referring now to FIGS. 5A-5C, some embodiments of the present disclosure are directed to a composition including a LMWH. In some embodiments, the LMWH is prepared via enzymatic conversion of LMW-NSNAH prepared from a bacterial source, e.g., E. coli CPS. In some embodiments, the LMWH in the composition meets USP enoxaparin specifications.

The chemoenzymatic synthesis of LMWH via a series of enzymatic modifications to LMW-NSNAH consistent with the embodiments of the present disclosure that meets the USP enoxaparin specifications was demonstrated. The conversion of NSNAH into NSNA2SH was catalyzed by C5-Epi, which converts GlcA residues into IdoA residues in a reversible reaction, that is then locked in place with 2-O-sulfation via 2-OST to afford IdoA2S residues. NSNA2SH was converted into NSNA2S6SH using 6-OST-1 and 6-OST-3. NSNA2S6SH was converted into LMWH using 3-OST. The C5-Epi/2-OST and 6-OST-X reactions were monitored by disaccharide compositional analysis. The 3-OST reaction was monitored by anti-Xa activity assay. Disaccharide compositional analysis was used for determining sulfation status, with a targeted range NS2S of 68-74% based on commercial enoxaparins.

Referring specifically to FIG. 5A, the conversion of NSNAH to NSNA2SH was determined at 4, 12, 24, 48, 72, 96 and 120 h time points. Without wishing to be bound by theory, the synthesis of the LMWH consistent with the embodiments of the present disclosure was much slower than the synthesis of chemobiosynthetic UFH due to reduced activity of these enzymes on shorter chain substrates. The maximum conversion percentage reached was 69.3% of NS2S at the 96 h time point, which met the USP enoxaparin specifications. Referring now to FIG. 5B, the conversion of NS2S to NS2S6S was completed in 24 h. UFHs have chains of sufficient length to bind both AT and thrombin to afford a ternary complex inactivating thrombin and thus preventing clot formation. In contrast, LMWH are comprised of smaller chains than UFH and most of these are of sufficient length for binding AT, inactivating factor Xa. Thus, the synthesis of the LMWH consistent with embodiments of the present disclosure was monitored through anti-Xa activity. Referring now to FIG. 5C, the potency of anti-factor Xa of enoxaparin is no less than 90 IU/mg and no more than 125 IU/mg on the dried basis. This activity could be reached after 120 h of treatment with 3-OST and there was no increased anticoagulant activity on further enzymatic reaction.

Referring again to FIG. 4B, GPC was used to determine the molecular weight of the LMWH consistent with embodiments of the present disclosure using USP enoxaparin sodium molecular weight calibrants. The USP criteria of weight average molecular weight for enoxaparin sodium is 4,500 Da, the range being between 3,800 and 5,000 Da. Since sulfation increases the molecular weight of the final product, a target molecular weight of 3,800-4,500 Da for the LMW-NSNAH intermediate was set. As expected, starting at 4,200 Da for low molecular weight NSNAH the molecular weight of the final LMWH product had increased to 4,350 Da.

The anticoagulant activity of the NSNA2S6SH intermediate and final LMWH product was measured using the methods described in the current USP enoxaparin monograph. The target anticoagulant activity of enoxaparin sodium has a potency of not less than 90 and not more than 125 anti-factor Xa International Units (IU)/mg, and not less than 20.0 and not more than 35.0 anti-factor Ila IU/mg, calculated on a dry basis. The ratio of anti-Xa to anti-Ila activity is between 3.3 and 5.3.

20 μL of reaction solution was periodically removed at various time points and anti-Xa activity and concentration analysis performed by HPLC-GPC. The anti-Xa activity (see FIG. 5C) increased in the first 48 h and then slowed down until reaching an activity of 105 IU/mg.

Referring to Table 1 below, the LMWH consistent with embodiments of the present disclosure had an anti-Xa of 105 IU/mg and 24 IU/mg of anti-Ila activity with anti Xa/Ila ratio of 4.4, consistent with USP enoxaparin.

TABLE 1
Summary of anticoagulant activity and IC₅₀ values
of LMWHs from triplicated preparations

| Anticoagulant activities | | | | IC₅₀ | |
|---|---|---|---|---|---|
| | Anti-Xa | Anti-IIa | antiXa/IIa | AT | PF4 |
| | (IU/mg) | (IU/mg) | ratio | (μg/mg) | (μg/mg) |
| Enoxaparin | 110 ± 4.4 | 28 ± 2.6 | 3.9 ± 0.22 | 11.0 ± 0.53 | 2.7 ± 0.26 |
| Chemobiocatalytic | 105 ± 2.6 | 24 ± 1.0 | 4.4 ± 0.13 | 12.0 ± 0.29 | 2.8 ± 0.35 |
| LMWH | | | | | |
| USP criteria | 90-125 | 20.0-35.0 | 3.3-5.3 | — | — |

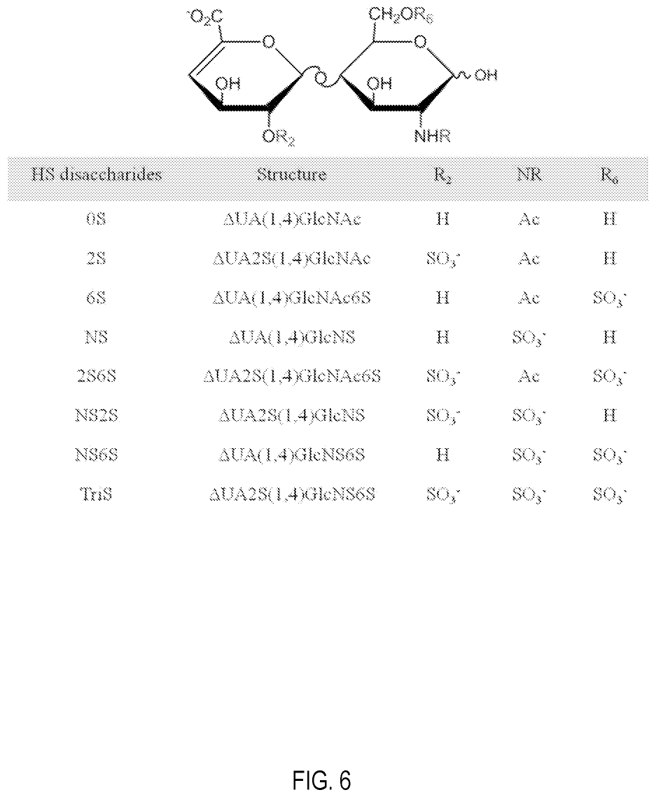

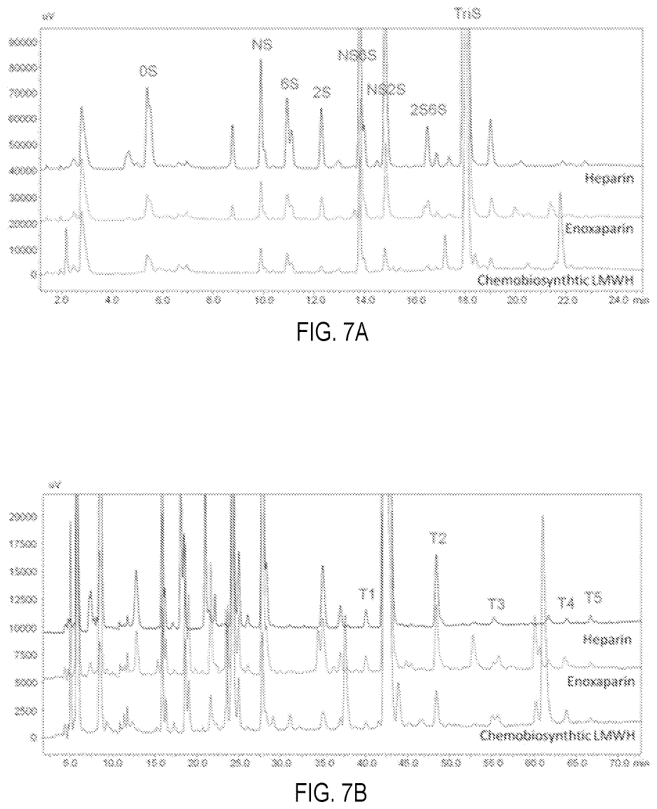

Referring now to FIG. 6, disaccharide compositional analysis of the LMWH consistent with embodiments of the present disclosure and its intermediates was performed utilizing treatment with heparin lyases I, II and III. These treatments afforded 8 different disaccharide products based on sulfation levels and positions. These disaccharides were then analyzed by strong anion exchange high-performance liquid chromatography (SAX-HPLC) to monitor the intermediates biosynthesis and final product (see FIGS. 7A-7B). The disaccharide compositions of heparin, enoxaparin controls, and LMWH consistent with embodiments of the present disclosure are shown in Table 2 below.

TABLE 2
Disaccharide and tetrasaccharide composition analysis
of LMWHs from triplicated preparations

| | Heparin control | Enoxaparin (%) | LMWH (%) |
|---|---|---|---|
| 0S | 4.2 ± 0.15 | 3.1 ± 0.28 | 3.9 ± 0.21 |
| NS | 3.3 ± 0.10 | 3.0 ± 0.35 | 3.8 ± 0.21 |
| 6S | 3.3 ± 0.12 | 3.6 ± 0.07 | 4.4 ± 0.14 |
| 2S | 2.0 ± 0.21 | 1.9 ± 0.07 | 0.1 ± 0.08 |
| NS6S | 10.6 ± 0.23 | 10.3 ± 0.49 | 17.3 ± 0.49 |
| NS2S | 7.7 ± 0.21 | 7.0 ± 0.14 | 3.5 ± 0.0 |
| 2S6S | 1.5 ± 0.23 | 1.8 ± 0.28 | 0.9 ± 0.39 |
| Tetra 1 | 0.3 ± 0.06 | 0.4 ± 0.08 | 0.3 ± 0.08 |
| TriS | 65.1 ± 0.81 | 66.3 ± 0.07 | 62.6 ± 0.01 |
| Tetra 2 | 1.7 ± 0.21 | 1.9 ± 0.17 | 1.8 ± 0.14 |
| Tetra 3 | 0.1 ± 0.05 | 0.4 ± 0.09 | 0.5 ± 0.09 |
| Tetra 4 | 0.1 ± 0.06 | 0.3 ± 0.02 | 0.6 ± 0.07 |
| Tetra 5 | 0.1 ± 0.03 | 0.1 ± 0.04 | 0.1 ± 0.04 |
| Tetra 1-5 subtotal | 2.3 ± 0.31 | 3.1 ± 0.40 | 3.3 ± 0.40 |
| Total (di & tetra) | 100.0 | 100.0 | 100.0 |

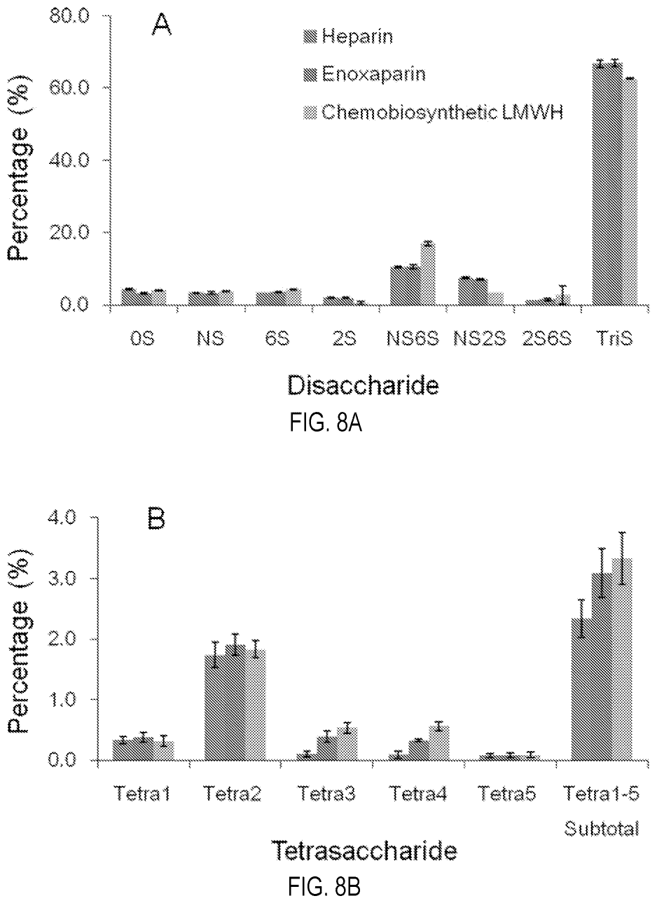

Referring now to FIGS. 8A-8B, treatment of heparosan isolated from the bacterial source with chemical N-sulfation, 2-OST/C5-Epi, 6-OST, and 3-OST treatment consistent with the embodiments described above, afforded a disaccharide composition that was similar to enoxaparin. The TriS content of the chemobiocatalytic LMWH was 62.6% compared to enoxaparin at 66.3%. The NS6S of the chemobiocatalytic LMWH was 17.3%, higher than enoxaparin of 10.3%, while NS2S of the LMWH was 3.5%, lower than 7.0% of enoxaparin. This suggests that the 2-OST conversion was lower than 6-OST conversion. It is noted that 3-O-sulfated glucosamine residues are resistant to heparin lyases cleavage. Thus, in addition to anticoagulant activity analysis, tetrasaccharide analysis was undertaken next by treating with heparinase I, II and III, followed by analysis of the resulting resistant tetrasaccharides with SAX-HPLC.

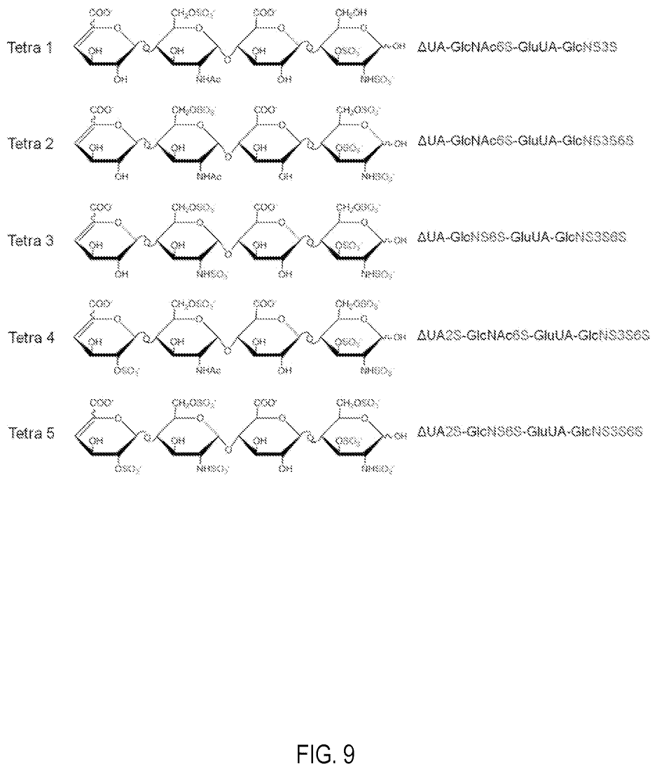

Referring now to FIG. 9, five 3-OST-including tetrasaccharides have been characterized: (1) ΔUA-GlcNAc6S-GlcUA-GlcNS3S (where ΔUA is deoxy-α-L-threo-hex-4-enopyranosyluronic acid); (2) ΔUA-GlcNAc6S-GlcUA-GlcNS3S6S; (3) ΔUA-GlcNS6S-GlcUA-GlcNS3S; (4) ΔUA2S-GlcNAc6S-GlcUA-GlcNS3S6S; (5) ΔUA2S-GlcNS6S-GlcUA-GlcNS3S6S. The results revealed that the chemobiocatalytic LMWH has a similar 3-OST-including tetrasaccharide distribution compared to enoxaparin (see again Table 2). The LMWH produced via embodiments of the present disclosure are highly close to enoxaparin in disaccharide and tetrasaccharide composition analysis.

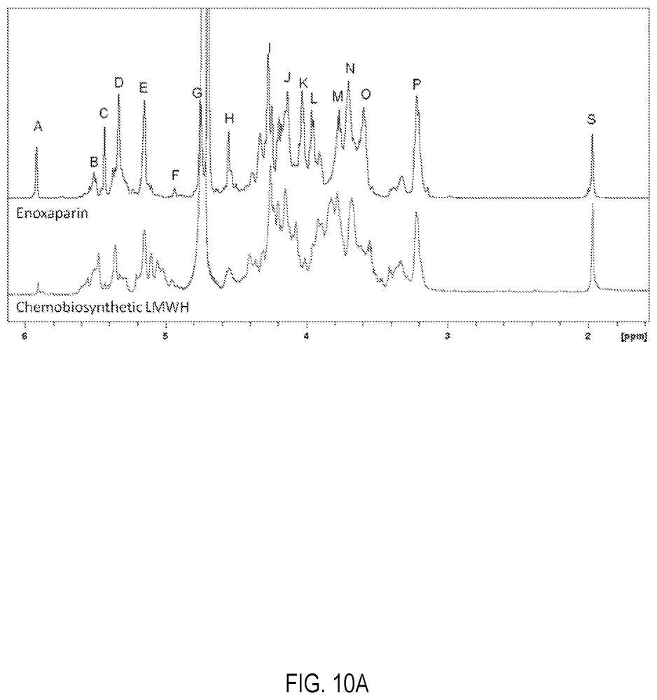

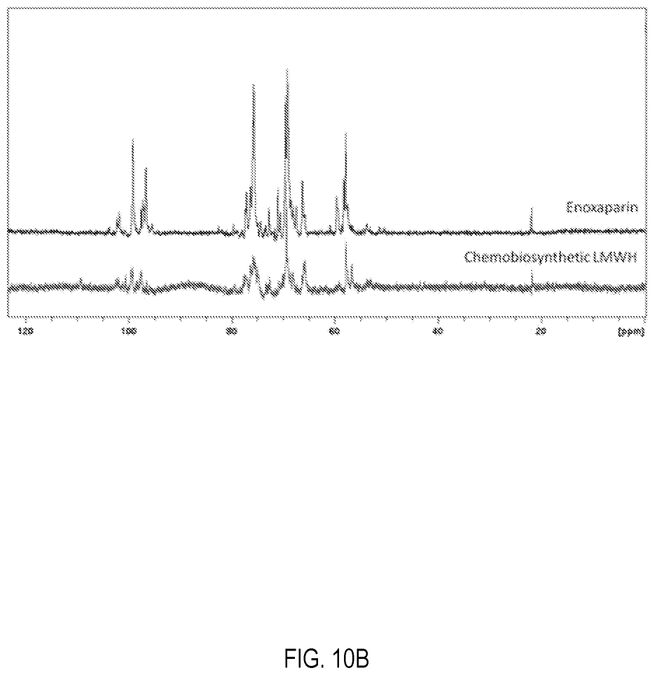

Referring now to FIGS. 10A-10B, one-dimensional ¹H and ¹³C NMR spectra were performed to characterize the structure of enoxaparin and LMWH produced via embodiments of the present disclosure. The enoxaparin ¹H peaks can be all assigned. The spectra of the two LMWHs looked quite similar but with some differences. The GlcNS3S peak overlapped H1 of ΔUA2S from 5.44 to 5.42 ppm. The IdoA2S peak was assigned from 5.17 to 5.09 ppm. The H4 ΔUA intensity at 5.90 ppm of chemobiocatalytic LMWH was lower compared to enoxaparin. The signal peaks at 5.48, 5.43, 5.33, 5.13, 5.07, and 4.51 ppm corresponded to the anomeric hydrogen. The LMWH had two more peaks from 5.09 to 5.00 ppm, which without wishing to be bound by theory, could be IdoA2S or impurities. In comparison to enoxaparin or generic enoxaparins, the chemobiocatalytic LMWH produced via embodiments of the present disclosure had very small amounts of 1,6-anhydromannose.

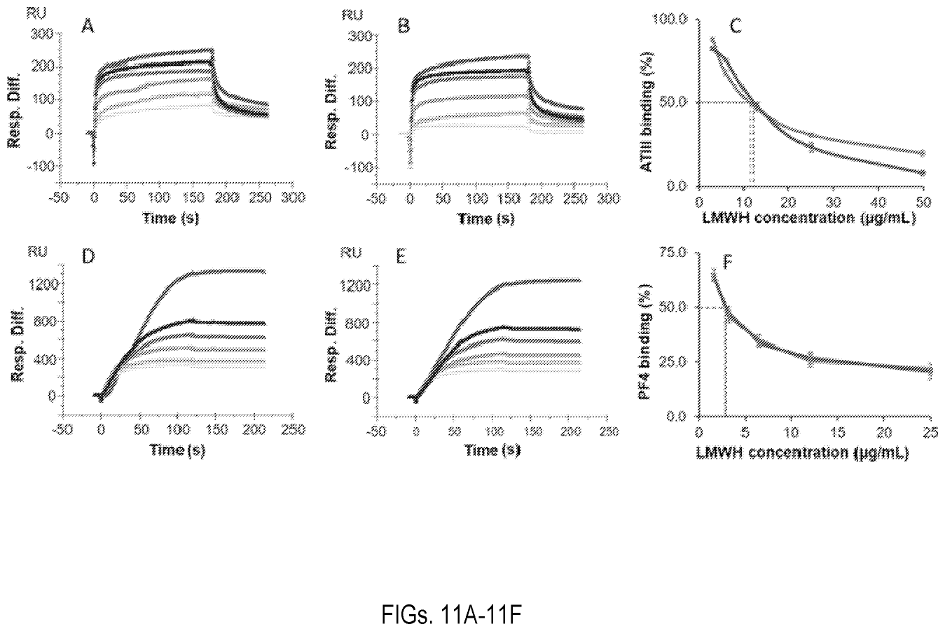

Referring now to FIGS. 11A-11C, the anticoagulant activity of heparin is primarily mediated through its binding and regulation of AT. Accordingly, the interaction between heparin and AT is a step for the anticoagulation process. Competition surface plasmon resonance (SPR) was used to measure the competitive AT binding of USP heparin immobilized on the chip surface vs. LMWH produced via embodiments of the present disclosure. The IC₅₀ values resulting in a 50% decrease in response units (RU) can be calculated from the plots over a range of the LMWH solution concentrations (up to 50 μg/mL). The results of IC₅₀ values for enoxaparin and chemobiocatalytic LMWH were 11.0 and 12.0 μg/mL, respectively. Hence, the AT binding activity of LMWH produced via embodiments of the present disclosure was slightly lower than enoxaparin, but still in an acceptance range.

Referring to FIGS. 11D-11F, of particular concern is heparin-induced thrombocytopenia (HIT) caused by interaction of heparin and platelet factor IV (PF4), resulting in an adverse immunological disorder. The analysis of HIT potential for LMWH produced via embodiments of the present disclosure was performed. A rapid method was used to evaluate the PF4 binding and calculate IC₅₀ value through solution competition SPR. The measured IC₅₀ for LMWH produced via embodiments of the present disclosure was 2.8 μg/mL compared to enoxaparin of 2.7 μg/mL. These results were comparable to LMWH samples ranging from 2.4 to 2.9 μg/mL. The binding affinity of the LMWH to PF4 is much smaller than UFH, resulting in a lower potential of HIT for LMWH.

### EXAMPLES

Materials. Escherichia coli K5 heparosan CPS was prepared through fermentation. The 2-, 6-, 3-OST and C5-Epi enzymes were prepared. Enoxaparin LMWH standard, enoxaparin sodium molecular weight calibrant A (1400, 2250, 4550 and 9250 Da) and B (1800, 3350 and 6650 Da) were purchased from the United States Pharmacopeia (USP, Rockville, MD). Human antithrombin III (AT) and platelet factor 4 (PF4) were purchased from Hyphen BioMed (Neuville-sur-Oise, France). Recombinant Flavobacterium heparinum heparin lyase I, II, III (EC Nots. 4.2.2.7, 4.2.2.X, and 4.2.2.8, respectively) were expressed in E. coli and purified. Unsaturated heparin disaccharide standards were purchased from Iduron (Manchester, UK). Biophen heparin anti-Xa (2 stages) and anti-IIa (2 stages) kits were purchased from Aniara (West Chester, OH, USA).

Removal of glycolipid terminus of K5 heparosan. The heparosan CPS prepared from E. coli K5 with fructosyl transferase removed was treated with hydrochloric acid to remove glycolipid acceptor 3-deoxy-D-manno-oct-2-ulosonic acid (Kdo). Heparosan was dissolved in hydrochloric acid solution and adjusted pH to 1, then incubated at 90° C. for 1 h. The solution was re-adjusted pH to 7 by sodium hydroxide and desalted by dialysis. The results were determined by NMR analysis.

Preparation of low molecular weight N-sulfo, N-acetyl heparosan (LMW-NSNAH) by chemical cleavage. De-N-acetylation and depolymerization of Kdo-free heparosan were undertaken by a controlled alkaline reaction. The sample (20 g/L) was dissolved in 50 mL of 2 N NaOH and incubated for 48 h at 65° C. in a shake flask, cooled to room temperature, and pH adjusted to 7.0 with HCl. Controlled re-acetylation was undertaken by adding methanol (3.5 mL), anhydrous sodium carbonate (130 mM/L), and acetic anhydride (53 μM/L each for four times with 20 min intervals). The amount of acetic anhydride was added to reach 10-15% of N-acetyl group as determined by NMR. The N-sulfation was next undertaken by adding an equal portion of anhydrous sodium carbonate (130 mM/L) and trimethylamine sulfur trioxide (76 mM/L), and mixed for 48 h at 47° C. The sulfation level was monitored by measuring unsubstituted amines using an o-phthaldialdehyde (OPA) assay. The sulfate and the acetyl group ratio were determined by NMR. The low molecular weight LMW-NSNAH was precipitated with 85% methanol at 4° C. overnight. The remaining salt was removed by washing four times with 85% methanol and centrifuged at 1800×g.

Preparation of chemobiosynthetic LMWH by enzymatic modifications. LMW-NSNAH sample (50 mg) was treated with C5-Epi and 2-OST to afford low molecular weight N-sulfo, N-acetyl, 2-sulfo heparosan (LMW-NSNA2SH). The detailed reaction conditions were as follows: substrate concentration of 1 mg/mL, PAPS concentration of 5 mM, each immobilized enzyme (C5-Epi/2-OST) at 1 mg/mL in a 50% slurry. The reaction was incubated in 50 mM 2-(N-morpholino) ethane sulfonic acid buffer (pH 7.2) with 0.05% NaN₃ and 125 mM NaCl for 120 h at 37° C. After the reaction was complete, the mixture was filtered to remove enzyme resin and dialyzed using 1 kDa molecular weight cut-off membrane tube against distilled water to remove salt and other small molecular impurities. Disaccharide compositional analysis was used to monitor and confirm the sulfation reaction. The controlled 6-OST and 3-OST reactions were next undertaken using immobilized enzymes to yield LMWH, i.e., chemobiocatalytic LMWH. The reaction conditions were similar to that used in the C5-Epi/2-OST reaction. Disaccharide composition analysis and anti-Xa activity assay were used to monitor the reaction status, respectively.

Molecular weight determination by Gel Permeation Chromatography (GPC). Molecular weights were determined by GPC-high performance liquid chromatography (HPLC) using enoxaparin sodium molecular weight calibrants. A guard column BioSuite 7.5×75 mm was used to protect two series connected analytical columns: Waters BioSuite™ 125, 5 μm HR SEC 7.8×300 mm column (Waters Corporation, Milford, MA). The mobile phase was 0.5 M lithium nitrate and the flow rate was set at 0.6 mL/min. The sample injection volume was 20 μL with concentration at 5 mg/mL.

Anticoagulant activity. The anticoagulant activities of the products were determined using BIOPHEN Heparin Anti-Xa (2 stages) and Anti-Ila (2 stages) kits following the protocols provided by the manufacturer. Briefly, AT (anti-Xa reagent 1 (r1)), factor Xa (r2), and factor Xa specific chromogenic substrate (r3) were used for anti-Xa activity, and AT (anti-IIa reagent 1 (R1)), human thrombin (R2), and factor IIa specific chromogenic substrate (R3) were used for anti-II activity. Each reagent was reconstituted with 1 mL of distilled water and shaken until fully dissolved. After a ⅕ dilution in the appropriate buffer (Tris-EDTA-NaCl-PEG, pH 8.4) for r1/R1 and r2/R2, distilled water was used for r3/R3 to restore the reagents immediately before use. Reference standards and dilute samples were prepared to the appropriate concentrations. Samples (40 μL) were added into a 96-well plate and incubated for 5 min at 37° C., 40 μL r1/R1 was added and mixed well and incubated for 2 min, 40 μL r2/R2 was next added and incubated for 2 min, and 40 μL r3/R3 was added last and incubated for another 2 min. The reactions were stopped by adding 80 μL of 50 mM acetic acid. The absorbance was then determined at 405 nm. The anti-Xa and anti-Ila activities were calculated using a standard curve of different concentration of enoxaparin standards.

Disaccharide and tetrasaccharide composition analysis. Disaccharide and tetrasaccharide composition were determined by strong anion exchange (SAX)-HPLC with ultraviolet detector performed on a Shimadzu™ LC-2030 system (Shimadzu, Kyoto, Japan). Samples (100 g) were exhaustively digested using a mixture of heparin lyase I, II and III (10 mU each) in digestion buffer (50 mM ammonium acetate including 2 mM calcium chloride, pH 7.0) at 37° C. for 2 h. The reaction was terminated by boiling for 10 min and the denatured enzymes were removed by centrifugation at 10000×g for 10 min. The supernatant concentrated at 1 μg/μL was analyzed by an HPLC system coupled with a Shimadzu™ LC-20 AD pump, CBM-20A controller, SIL-20AHT auto-sampler, and SPD-20AV UV detector. A Spherisorb SAX chromatography column (4.0×250 mm, 5.0 μm, Waters) was equilibrated with mobile phase A (1.8 mM monobasic sodium phosphate, pH=3) and followed with gradient elution using mobile phase B (1.8 mM monobasic sodium phosphate with 2 M sodium perchlorate, pH=3). Disaccharide analysis used a gradient of mobile phase B that increased from 5% to 50% in 30 min, held for 5 min, then changed to 5% and held for 15 min. Tetrasaccharide analysis used a gradient of 15-32.5% mobile phase B from 0-40 min, 42.5% mobile phase B at 50 min, 50% at 54 min, and maintained for 1 min at a flow rate of 0.45 mL/min.

Nuclear magnetic resonance (NMR) spectroscopy analysis. The NMR spectra were obtained on a Bruker 800 MHz (18.8 T) standard-bore NMR spectrometer equipped with a ¹H/²H/¹³C/¹⁵N cryoprobe with z-axis gradients. Sample was dissolved in 0.4 mL of 99.96% D₂O and lyophilized, and then repeated twice. A ¹H/¹³C 1D NMR was carried out at 298 k.

Surface plasmon resonance (SPR) analysis. SPR measurements were performed on a BIAcore™ 3000 instrument (GE, Uppsala, Sweden) operated using BIAcore 3000 control and BIAevaluation™ software (version 4.0.1). Biotinylated heparin prepared by conjugating the reducing end of heparin to amine-PEG3-Biotin (Pierce, Rockford, IL) was immobilized to a streptavidin-coated chip based on the manufacturer's protocol. Competition studies between surface heparin and LMWH (produced via embodiments of the present disclosure) binding to proteins were performed using SPR through measurement of IC₅₀. AT (250 nM) or PF4 (125 nM) mixed with different concentrations of LMWHs in HBS-EP buffer (0.01 M 4-(2-hydroxyethyl)-1-piperazineethanesulfonic acid) (HEPES), 0.15 M NaCl, 3 mM ethylenediaminetetraacetic acid (EDTA), 0.005% surfactant P20, pH 7.4) were injected over the chip at a flow rate of 30 μL/min. Dissociation and regeneration were performed using sequential injection with 10 mM glycine-HCl (pH 2.5) and 2 M NaCl to obtain fully regenerated surface after each run. For each set of competition experiments, a control experiment was performed to ensure that the surface was completely regenerated, and the results obtained between runs were comparable.

Methods and systems of the present disclosure are advantageous to produce a LMWH suitable for equivalent use to enoxaparin sodium, the most widely used low molecular weight heparin product. The chemobiocatalytic LMWH of the present disclosure is intended to serve as a comparable version of traditional pharmaceutical LMWH.

Enoxaparin is typically obtained by alkaline depolymerization of heparin benzyl ester isolated exclusively from porcine intestinal mucosa. However, there are significant disadvantages to the preparation and use of porcine-derived enoxaparin, namely the variability of animal-sourced heparin starting material, the limited availability and poor control of source materials, and their impurities.

The LMWH of the present disclosure and compositions including that LMWH are prepared without the use of porcine-derived heparin, and thus prepared without a depolymerization step from porcine sourced UFH. Instead, methods of the present disclosure utilize bacterial sources, such as engineered E. coli K5, to generate heparosan for use as backbone precursor to an LMWH product. A depolymerization method, e.g., via an alkali composition, can obtain the appropriate chain length backbone, which can then be modified via C5-Epi, 2-O-, 6-O-, and 3-O-sulfotransferases. These methods are less expensive, yet prepare high purity, heterogeneous, polydisperse forms of enoxaparin. Further, such chemoenzymatically synthesized LMWHs have several advantages over LMWHs prepared from animal sourced UFH, including better source material availability, better control of manufacturing processes, reduced concerns about contamination, adulteration or animal virus, or impurities.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method of making low molecular weight heparin (LMWH), comprising:
providing an amount of heparosan;
contacting the heparosan with one or more acids to form acid-treated heparosan;
converting the acid-treated heparosan by depolymerization and de-N-acetylation thereof to form low molecular weight N-sulfo, N-acetyl heparosan (LMW-NSNAH); and
enzymatically converting the LMW-NSNAH to LMWH,
wherein the heparosan is an E. coli capsular polysaccharide.

2. The method according to claim 1, wherein the heparosan is synthesized via an engineered strain of E. coli K5.

3. The method according to claim 1, wherein contacting the heparosan with one or more acids to form acid-treated heparosan includes:
removing 3-deoxy-D-manno oct-2-ulosonic acid (Kdo) residues from the heparosan via acid hydrolysis.

4. The method according to claim 1, wherein converting the acid-treated heparosan to LMW-NSNAH includes hydrolysis of the acid-treated heparosan via:
treatment of the acid-treated heparosan with one or more bases;
treatment of the acid-treated heparosan with one or more additional acids;
contacting the acid-treated heparosan with one or more enzymes; or
combinations thereof.

5. The method according to claim 1, wherein converting the acid-treated heparosan by depolymerization and de-N-acetylation thereof to LMW-NSNAH further includes:
re-acetylating the acid-treated heparosan after de-N-acetylation thereof; and
N-sulfating the acid-treated heparosan to obtain LMW-NSNAH.

6. The method according to claim 5, wherein:
re-acetylating the acid-treated heparosan after de-N-acetylation thereof includes:
contacting the acid-treated heparosan with acetic anhydride; and
N-sulfating the acid-treated heparosan to obtain LMW-NSNAH includes:
contacting the acid-treated heparosan with trimethylamine sulfur trioxide, pyridine sulfur trioxide, or combination thereof.

7. The method according to claim 5, wherein re-acetylating the acid-treated heparosan after de-N-acetylation thereof includes:
contacting the acid-treated heparosan with about 53 μM/L acetic anhydride.

8. The method according to claim 5, wherein N-sulfating the acid-treated heparosan to obtain LMW-NSNAH further comprises:
contacting the acid-treated heparosan with about 76 mM/L trimethylamine sulfur trioxide.

9. The method according to claim 1, wherein enzymatically converting the LMW-NSNAH to LMWH further comprises:
contacting the LMW-NSNAH with C5-Epi and 2-OST to form N-sulfo, N-acetyl, 2-O-sulfo IdoA-including heparosan (NSNA2SH).

10. The method according to claim 9, wherein enzymatically converting the LMW-NSNAH to LMWH further comprises:
contacting the NSNA2SH with 6-O-sulfotransferase-3, 6-O-sulfotransferase-1, or combinations thereof to form N-sulfo, N-acetyl, 2-O-sulfo, 6-O-sulfo IdoA-including heparosan (NSNA2S6SH).

11. The method according to claim 10, wherein enzymatically converting the LMW-NSNAH to LMWH further comprises:
contacting the NSNA2S6SH with 3-O-sulfotransferase-1 to form LMWH.

12. The method according to claim 1, wherein the LMW-NSNAH has a molecular weight between about 3,800 and about 4,500 daltons.

13. The method according to claim 1, wherein the LMW-NSNAH includes between about 10% to about 15% N-acetyl groups.

14. The method according to claim 1, wherein the LMWH is a heterogeneous, polydisperse form of enoxaparin with an anti-factor Xa between about 90 and about 125 IU/mg.

15. An intermediate low molecular weight N-sulfo, N-acetyl heparosan (LMW-NSNAH), the LMW-NSNAH being produced by a method including:
providing an amount of heparosan, wherein the heparosan is an E. coli capsular polysaccharide;
contacting the heparosan with one or more acids to remove 3-deoxy-D-manno oct-2-ulosonic acid (Kdo) residues from the heparosan via hydrolysis to form acid-treated heparosan; and
converting the acid-treated heparosan by depolymerization and de-N-acetylation thereof to form LMW-NSNAH;
wherein the LMW-NSNAH has a molecular weight and a ratio of N-sulfo groups and N-acetyl groups such that enzymatic treatment thereof with a C5-epimerase and at least one sulfotransferase yields a final product with molecular weight and chemical properties consistent with animal-derived enoxaparin.

16. The intermediate LMW-NSNAH according to claim 15, wherein the LMW-NSNAH has a molecular weight between about 3,800 and about 4,500 daltons.

17. The intermediate LMW-NSNAH according to claim 15, wherein the LMW-NSNAH includes between about 10% to about 15% N-acetyl groups.

18. The intermediate LMW-NSNAH according to claim 15, wherein converting the acid-treated heparosan to LMW-NSNAH includes hydrolysis via:
treatment of the acid-treated heparosan with one or more bases;
treatment of the acid-treated heparosan with one or more additional acids;
contacting the acid-treated heparosan with one or more enzymes; or
combinations thereof.

19. The intermediate LMW-NSNAH according to claim 15, wherein converting the acid-treated heparosan by depolymerization and de-N-acetylation thereof to LMW-NSNAH further includes:
adding the acid-treated heparosan to a reaction medium including methanol, anhydrous sodium carbonate, and about 53 μM/L acetic anhydride to form re-acetylated heparosan; and
adding the re-acetylated heparosan to a reaction medium including anhydrous sodium carbonate and about 76 mM/L trimethylamine sulfur trioxide to obtain LMW-NSNAH.

20. A composition including a low molecular weight heparin (LMWH), wherein the LMWH is prepared via enzymatic conversion of low molecular weight N-sulfo, N-acetyl heparosan (LMW-NSNAH) prepared from E. coli capsular polysaccharide.
